(19) United States
(12) Patent Application Publication
(10) Pub. No.: US 2017/0014283 A1
(43) Pub. Date: Jan. 19, 2017

(54) Incontinence Sack

(71) Applicant: Desiree Wright, Uniondale, NY (US)

(72) Inventor: Desiree Wright, Uniondale, NY (US)

(21) Appl. No.: 14/800,858

(22) Filed: Jul. 16, 2015

(51) Int. Cl. A61F 13/72; A61F 5/44; A61F 13/70

(52) U.S. Cl. CPC A61F 13/72; A61F 13/70; A61F 5/4401; A61F 2013/15121

(57) ABSTRACT

An incontinence sack includes an undergarment configured to fit about a waist and legs of a male person, the undergarment having an inside front portion; a pouch defining a pouch pocket and attached to the harness and positioned on the inside front portion of the undergarment and an absorbent sack defining an absorbent sack pocket and positionable within the pouch, wherein the absorbent sack pocket is configured to accept a distal end of a penis of the male person.

### BACKGROUND

The present invention relates to a device for male incontinence.

Incontinence affects men for many reasons, for example, age and medical conditions. Whatever the cause, many men experience uncontrolled urine discharge from the penis. This urine discharge often proves uncomfortable and embarrassing.

Commonly, medical providers prescribe the use of a standard adult diaper to absorb the urine discharge and thus prevent discomfort for the patient. These diapers often come in various shapes and sizes to provide additional comfort to the wearer.

Although these prior art solutions are available, many men are uncomfortable wearing a diaper to collect the urine discharge. Some of this discomfort comes from wearing an item that a man might think is not masculine.

What is needed is a male incontinence device that provides adequate absorbance and is comfortable to wear.

### SUMMARY

Provided herein is an incontinence sack. The incontinence sack according to the present disclosure it both functional in that is provides adequate absorbance for incontinence discharge and is also comfortable to wear.

According to the present disclosure, an incontinence sack is provided. The incontinence sack includes an undergarment having a pouch and an absorbent sack included in the pouch.

According to the present disclosure, an incontinence sack includes an undergarment configured to fit about a waist and legs of a male person, the undergarment defining an inside front portion; a pouch defining a pouch pocket and attached to the undergarment and positioned on the inside front portion of the undergarment; and an absorbent sack defining an absorbent sack pocket and positionable within the pouch, wherein the absorbent sack pocket is configured to accept a distal end of a penis of the male person.

According to the present disclosure, an absorbent sack for use in an incontinence sack includes a posterior part; and an anterior part attached to the posterior part, wherein the posterior part of the absorbent sack and anterior part of the absorbent sack define an absorbent sack pocket, wherein the absorbent sack pocket is configured to accept a distal end of a penis of a male person.

According to the present disclosure, an incontinence kit includes an undergarment configured to fit about a waist and legs of a male person, the undergarment having an inside front portion; a pouch defining a pouch pocket and attached to the undergarment and positioned on the inside front portion of the undergarment; and a plurality of absorbent sacks, each absorbent sack defining an absorbent sack pocket and positionable within the pouch, wherein the absorbent sack pocket is configured to accept a distal end of a penis of the male person.

### BRIEF DESCRIPTION OF THE DRAWINGS

Various embodiments are described below with reference to the drawings wherein:

FIG. 1 is a perspective view of an incontinence sack according to the present disclosure;

FIG. 2 is a perspective view of an incontinence sack with an absorbent sack partially inserted into a harness pouch according to the present disclosure;

FIG. 3 is a perspective view of an incontinence sack with an absorbent sack fully inserted into a harness pouch according to the present disclosure;

FIG. 4 is a perspective view of an incontinence sack fitted on a male patient according to the present disclosure;

FIG. 5 is a cross sectional view of an incontinence sack with an absorbent sack fully inserted into a harness pouch according to the present disclosure;

FIG. 6 is a side plan view of an incontinence sack with an absorbent sack fully inserted into a harness pouch according to the present disclosure;

FIG. 7 is a perspective view of an incontinence sack fitted on a distal end of a penis of a male patient according to the present disclosure;

FIG. 8 is a posterior plan view of an absorbent sack according to the present disclosure;

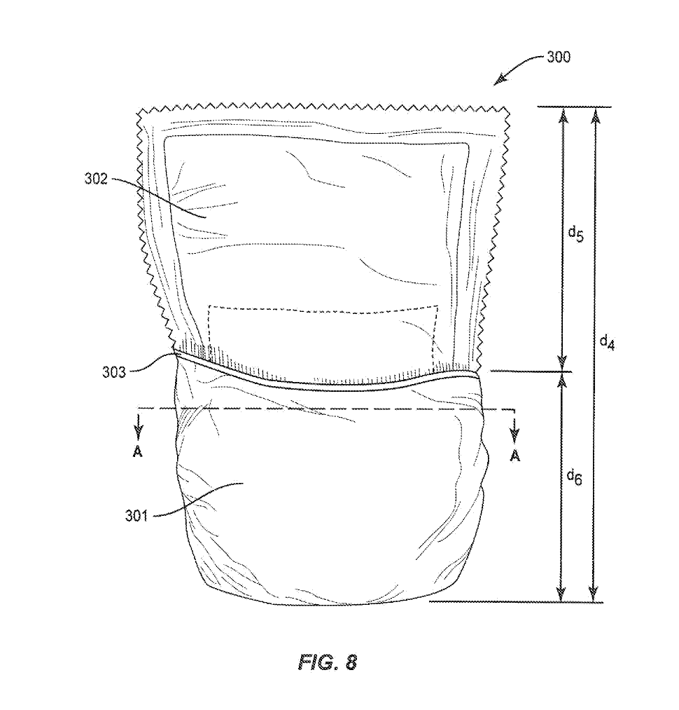

FIG. 9 is a side plan view of an absorbent sack according to the present disclosure;

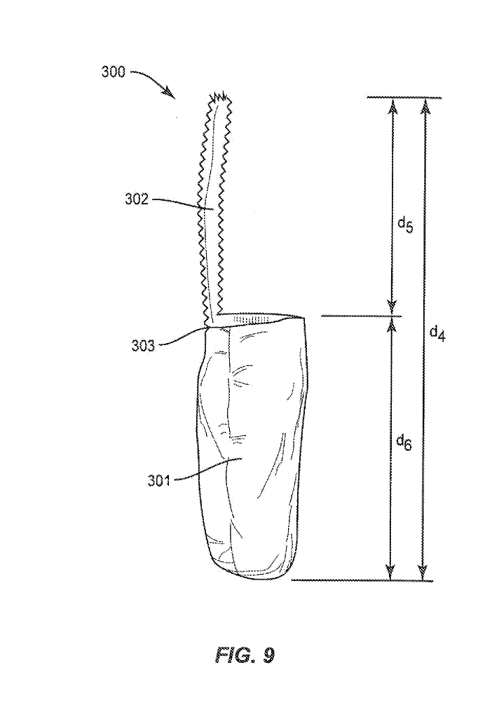

FIG. 10 is an anterior plan view of an absorbent sack according to the present disclosure; and

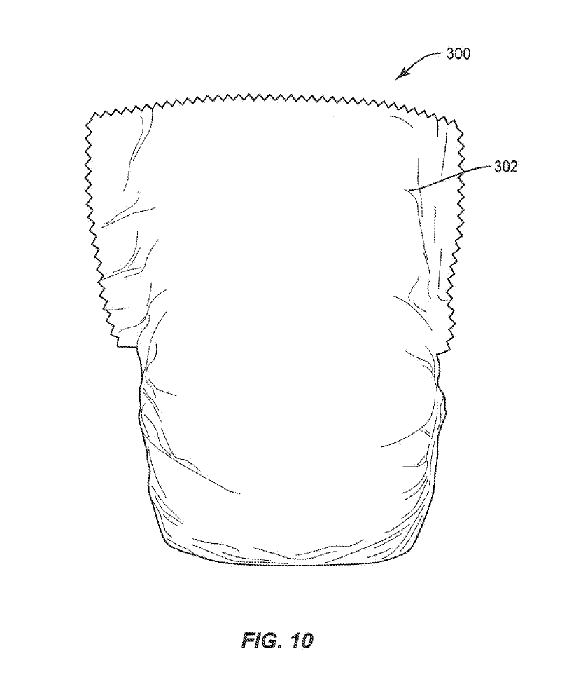

FIG. 11 is a cross sectional view of an absorbent sack according to the present disclosure.

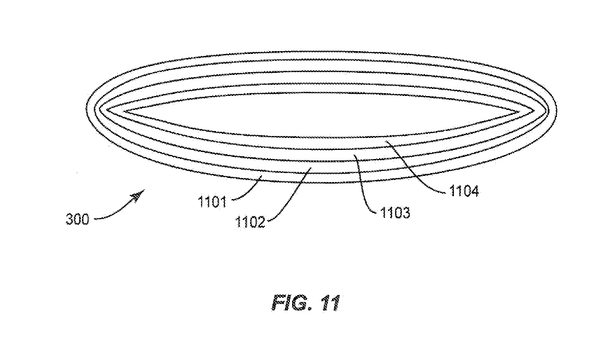

Like reference numerals indicate similar parts throughout the figures.

### DETAILED DESCRIPTION OF PREFERRED EMBODIMENT(S)

The present disclosure may be understood more readily by reference to the following detailed description of the disclosure taken in connection with the accompanying figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure.

Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

Reference will now be made in detail to the exemplary embodiments of the present disclosure, which are illustrated in the accompanying drawings.

The present invention is an incontinence sack 100. Incontinence sack 100 includes an undergarment 200 and an absorbent sack 300. Although the incontinence sack according to the present disclosure is described as utilizing a harness, other configurations are contemplated. For example, the incontinence sack according to the present disclosure can, in place of the harness, include undershorts that include the pouch for accepting the absorbent sack. The undershorts can be of the sports variety for a close and tight fit.

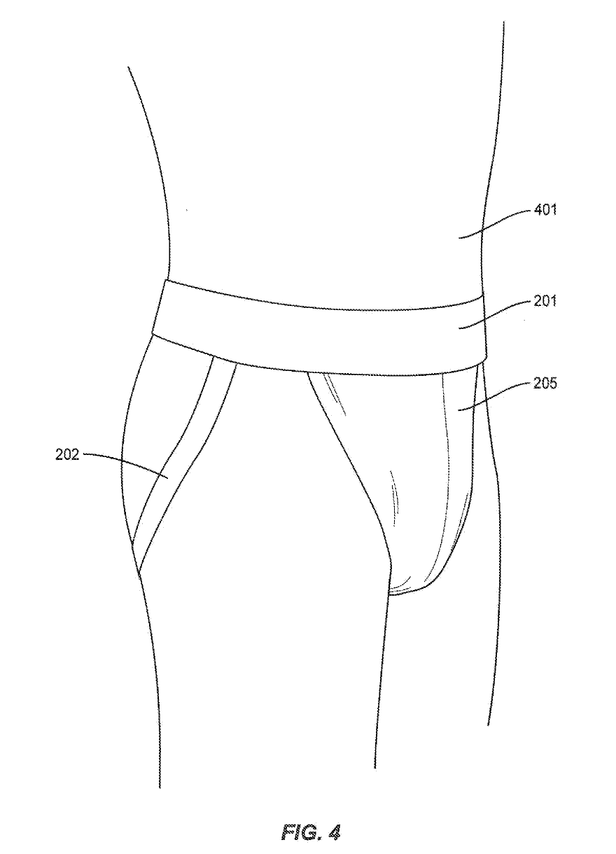

Harness 200 includes a waist strap 201, leg straps 202 and a pouch 203. Waist strap 201 and leg straps 202 are configured to fit about a lower part of a male torso 401. (See FIG. 4.) The configuration of waist strap 201 and leg straps 202 can vary to accommodate different sizes of male torsos. Waist strap 201 and leg straps 202 can be made from a variety of materials including but not limited to, cloth or silk, and may or may not be made of materials that stretch for a more comfortable fit. Adjustable straps are also contemplated.

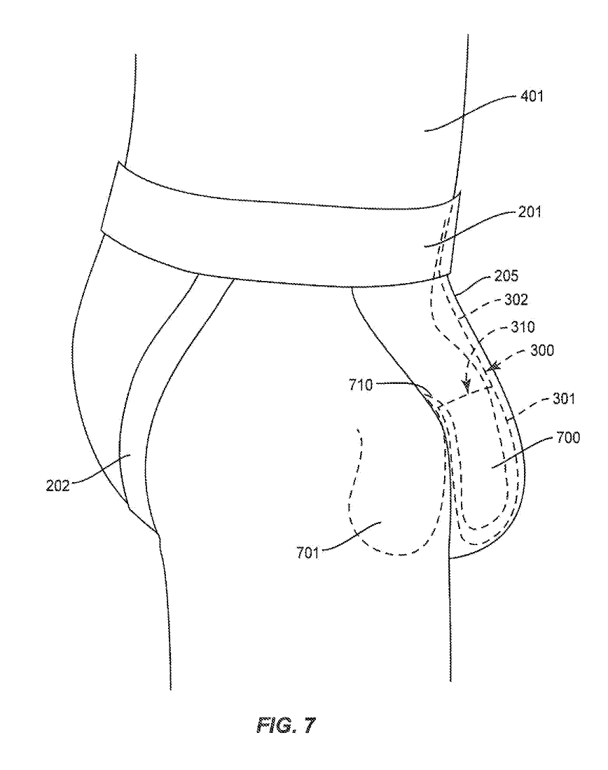

Pouch 203 includes a pouch posterior portion 204 and a pouch anterior portion 205. Pouch posterior portion 204 defines a pouch posterior upper edge 207 and pouch anterior portion 205 defines a pouch anterior upper edge 208. Pouch 203 is affixed to waist strap 201 and leg straps 202 as shown with pouch anterior portion 205 affixed to waist strap 201. Pouch 203 is affixed to leg straps 202 at or about a lower point where pouch posterior portion 204 joins with pouch anterior portion 205. Pouch posterior upper edge 207 is configured lower than pouch anterior upper edge 208. Pouch posterior portion 204 and pouch anterior portion 205 define a pouch pocket 210. This configuration of pouch posterior upper edge 207 and pouch anterior upper edge 208 provide for a comfortable fit of pouch 203 on to the penis in contrast to prior art incontinence devices. (See, FIG. 7.) Although pouch 203 is described as comprising 2 elements, i.e., pouch posterior portion 204 and pouch anterior portion 205, pouch 203 can be manufactured as a single pocket-forming element.

Harness 200 can include a Velcro strip 206 as an optional means for securing absorbent sack 300 to harness 200. Other means are contemplated, including, but not limited to, snaps, clips, tape, etc. In addition, the means for securing absorbent sack 300 to harness 200 can be positioned on absorbent sack 300, or may be a configuration of two parts, one of which is on harness 200 and another on absorbent sack 300.

Absorbent sack 300 includes an absorbent sack posterior portion 301 and an absorbent sack anterior portion 302. Absorbent sack posterior portion 301 defines an absorbent sack posterior upper edge 303 and absorbent sack anterior portion 302 defines an absorbent sack anterior upper edge 304. Absorbent sack posterior upper edge 303 is configured lower than absorbent sack anterior upper edge 304. Absorbent sack posterior portion 301 and absorbent sack anterior portion 302 define an absorbent sack pocket 310. This configuration of absorbent sack posterior upper edge 303 and absorbent sack anterior upper edge 304 provide for a comfortable fit of absorbent sack 300 on to the penis in contrast to prior art incontinence de ices. (See, FIG. 7.) Although absorbent sack 300 is described as comprising 2 elements, i.e., absorbent sack posterior portion 301 and absorbent sack anterior portion 302, absorbent sack 300 can be manufactured as a single pocket-forming element.

Absorbent sack 300 is manufactured from materials that absorb liquid, in particular, urine, and prevent leakage through the outside of the absorbent sack 300. These materials are well known in the child and adult diaper industries. Generally, absorbent sack 300 includes an outer layer 1102 preferably made from moisture impermeable or liquid resistant material (e.g. a polypropylene film) and an inner layer 1103 preferably formed from liquid absorbent materials, fluff materials, cellulose wading, absorbent cotton fibers, polyester of polyolefin mixed combination, or combinations thereof. Other materials for the outer and inner layers are contemplated. Additional layers of materials are also contemplated, for example an outer layer 1101 of soft material for comfort and/or an inner layer 1104 of soft and moisture permeable material also for comfort.

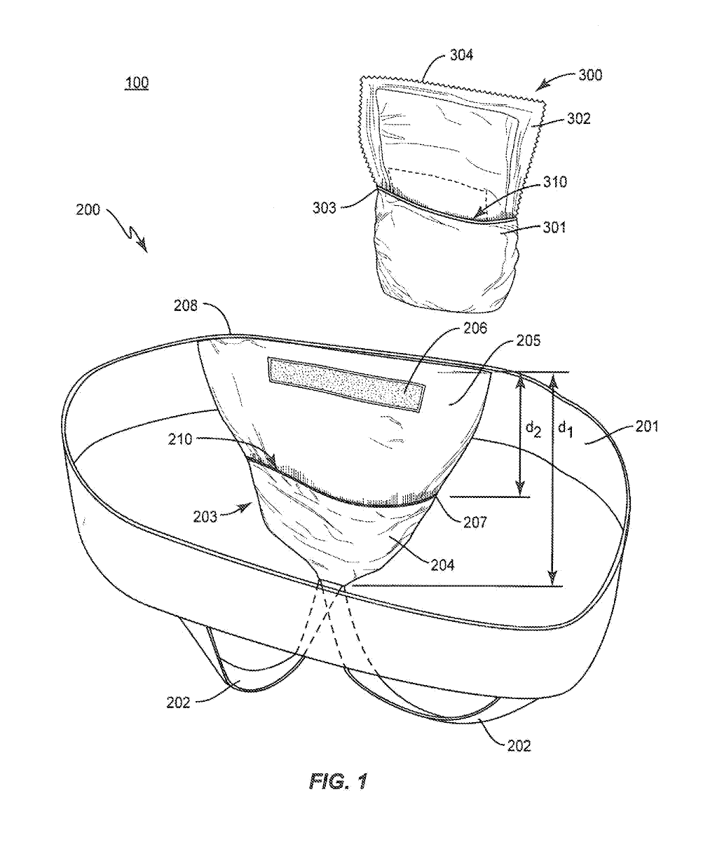

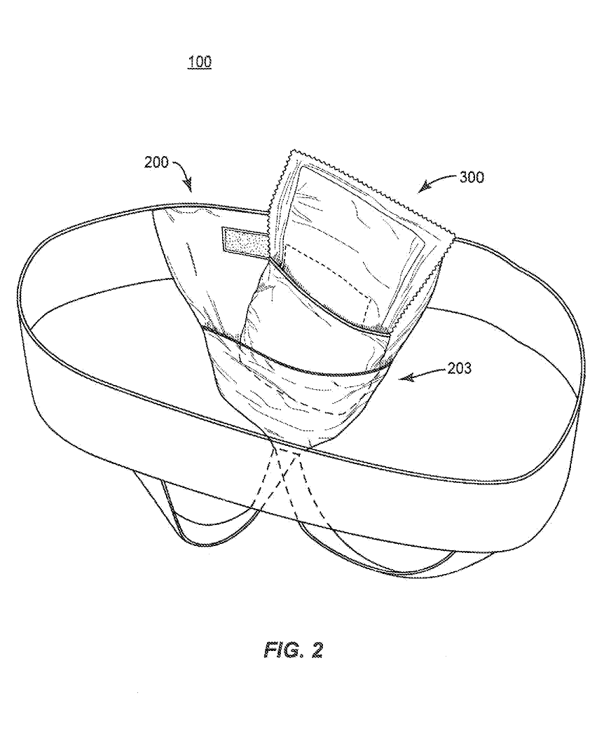

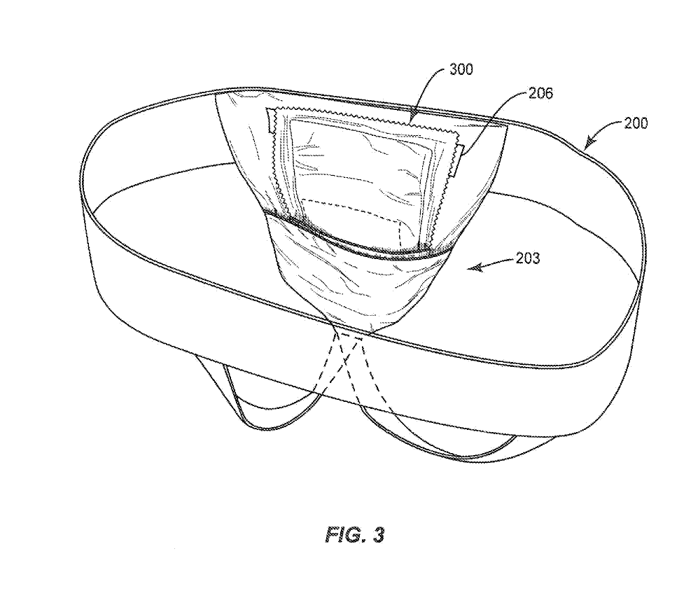

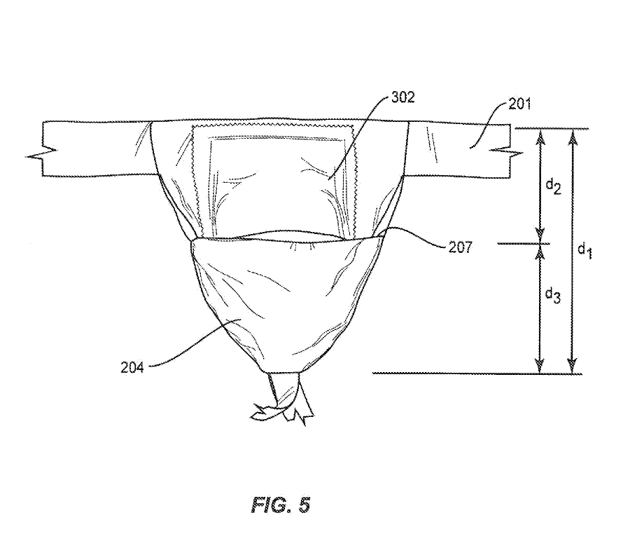

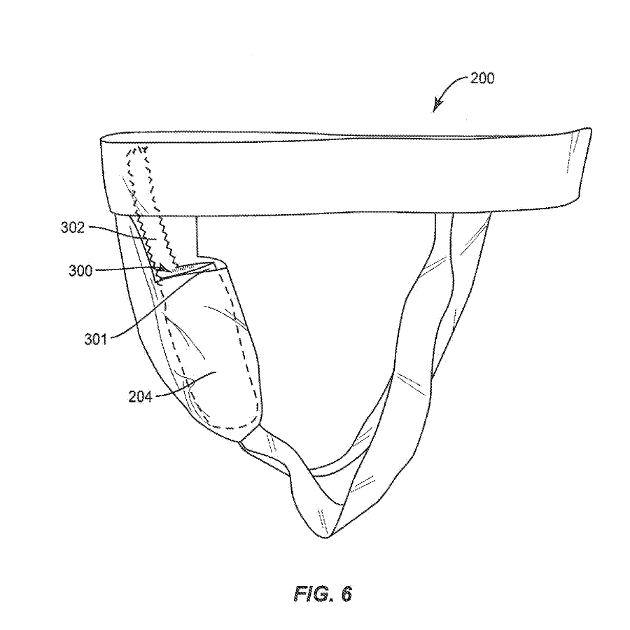

As described above, pouch 203 and absorbent sack 300 are each designed to create a respective pocket 210/310. Pouch posterior upper edge 207 is configured lower than pouch anterior upper edge 208 and absorbent sack posterior upper edge 303 is configured lower than absorbent sack anterior upper edge 304. Pouch anterior portion 205 defines an overall length d1 of pouch 203, a length d2 is defined from pouch anterior upper edge 208 and pouch posterior upper edge 207, and a length d3 defines a depth of pouch pocket 210. Absorbent sack anterior portion 302 defines an overall length d4 of absorbent sack 300, a length d5 is defined from absorbent sack anterior upper edge 304 and absorbent sack posterior upper edge 303, and length d6 defines a depth of absorbent sack pocket 310. When absorbent sack 300 is inserted into pouch 203 (see, e.g., FIGS. 1-3), pouch posterior upper edge 207 substantially aligns with absorbent sack posterior upper edge 303 and pouch anterior upper edge 208 substantially aligns with absorbent sack anterior upper edge 304. (See, e.g., FIGS. 3, 5 and 6.)

As shown in FIG. 7, having posterior edges 207/303 lower than anterior edges 208/304 of pouch 203 and absorbent sack 300 allows penis 700 to be positioned into pouch 310 in a comfortable and relaxed manner. Pocket 310 is designed deep enough (e.g. depth d6) to accommodate the distal end of penis 700 and snug enough to maintain penis 700 within pocket 310 during normal use and activity. Absorbent sack posterior upper edge 303 is preferably positioned lower that the point 710 where penis 700 meets with scrotum 701 when harness 200 is positioned on the body. Lengths d1-d6 can be varied to accommodate different size patients as needed.

Absorbent sack 300 is a removable and replaceable component of incontinence sack 100. After a use, absorbent sack 300 is removed from pouch 203 and replaced with a new and dry absorbent sack 300. It is contemplated that harness 200 is made from washable materials and can be used over and over again with only the need to replace absorbent sack 300 as necessary.

An incontinence sack kit is also contemplated. The kit can include a harness 200 and a plurality of absorbent sacks 300.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

1. An incontinence sack, comprising:
an undergarment configured to fit about a waist and legs of a male person and defining an inside front portion;
a pouch defining a pouch pocket and attached to the undergarment and positioned on the inside front portion of the undergarment; and
an absorbent sack defining an absorbent sack pocket and positionable within the pouch, the absorbent sack comprising:
an absorbent sack posterior part having an upper edge; and
an absorbent sack anterior part having an upper edge, the absorbent sack pocket is configured to accept a distal end of a penis of the male person, the upper edge of the absorbent sack posterior part is inferior to the upper edge of the absorbent sack anterior part, and the upper edge of the absorbent sack posterior part is configured to be positioned below a meeting point of the penis and a scrotum of the male patient.

2. The incontinence sack of claim 1, wherein the undergarment is configured as one of a harness or shorts.

3. The incontinence sack of claim 1, wherein the pouch comprises:
a pouch posterior part having an upper edge; and
a pouch anterior part having an upper edge,
wherein the upper edge of the pouch posterior part is inferior to the upper edge of the pouch anterior part.

4. (canceled)

5. (canceled)

6. The incontinence sack of claim 1, wherein the absorbent sack is removably attachable within the pouch.

7. The incontinence sack of claim 6, further comprising means for attaching the absorbent sack within the pouch.

8. The incontinence sack of claim 1, wherein the absorbent sack is disposable.

9. The incontinence sack of claim 1, wherein the undergarment and the pouch are manufactured from washable materials.

10. The incontinence sack of claim 1, wherein the absorbent sack comprises:
an outer moisture impermeable layer; and
an inner moisture absorbent layer.

11. The incontinence sack of claim 10, wherein the absorbent sack further comprises a moisture permeable inner layer of soft material.

12. An absorbent sack for use in an incontinence sack, comprising:
a posterior part; and
an anterior part attached to the posterior part,
wherein the posterior part of the absorbent sack and anterior part of the absorbent sack define an absorbent sack pocket,
wherein the absorbent sack pocket is configured to accept a distal end of a penis of a male person.

13. The absorbent sack of claim 12, wherein the absorbent sack comprises:
an absorbent sack posterior part having an upper edge; and
an absorbent sack anterior part having an upper edge,
wherein the upper edge of the absorbent sack posterior part is inferior to the upper edge of the absorbent sack anterior part.

14. The absorbent sack of claim 12, further comprising:
an outer moisture impermeable layer; and
an inner moisture absorbent layer.

15. The absorbent sack of claim 14, further comprising a moisture permeable inner layer of soft material.

16. An incontinence kit, comprising:
an undergarment configured to fit about a waist and legs of a male person, the undergarment having an inside front portion;
a pouch defining a pouch pocket and attached to the harness and positioned on the inside front portion of the undergarment; and
a plurality of absorbent sacks, each absorbent sack defining an absorbent sack pocket and positionable within the pouch, each absorbent sacks comprising:
an absorbent sack posterior part having an upper edge, and
an absorbent sack anterior part having an upper edge, the absorbent sack pocket is configured to accept a distal end of a penis of the male person, the upper edge of the absorbent sack posterior part is inferior to the upper edge of the absorbent sack anterior part, and the upper edge of the absorbent sack posterior part is configured to be positioned below a meeting point of the penis and a scrotum of the male patient.

17. The incontinence kit of claim 16, wherein the pouch comprises:
a pouch posterior part having an upper edge; and
a pouch anterior part having an upper edge,
wherein the upper edge of the pouch posterior part is inferior to the upper edge of the pouch anterior part.

18. (canceled)

19. (canceled)

20. The incontinence kit of claim 16, wherein the undergarment is one of a harness or shorts.
